(19) United States
(12) Patent Application Publication
(10) Pub. No.: US 2004/0241655 A1
(43) Pub. Date: Dec. 2, 2004

(54) Conditional touchdown multiplex polymerase chain reaction

(76) Inventors: Yuchi Hwang, Wugu Shiang (TW); Yun-Yun Cheng, Wugu Shiang (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/446,940

(22) Filed: May 29, 2003

(51) Int. Cl. C12Q 1/68; C12P 19/34

(52) U.S. Cl. 435/6; 435/91.2

(57) ABSTRACT

A high-throughput and cost-effective method for simultaneous amplification of target DNA sequences with high fidelity and workable rate is achieved by a two-stage amplification incorporating multiplex PCR with conditional touchdown strategies. This improved multiplex PCR comprises a simultaneous PCR and a specific PCR, and either one or both of the amplification steps are performed with a touchdown strategy, of which loose touchdown strategy is applied with a temperature lower than the optimized annealing temperature, and stringent touchdown strategy is applied with a temperature higher than the optimized annealing temperature.

### FIELD OF THE INVENTION

The present invention is directed to a high-throughput and cost-effective method for simultaneous amplification of multiple target DNA sequences with high fidelity and workable rate. And more specifically, the present invention relates to a multiplex polymerase chain reaction (MPCR) incorporating conditional touchdown strategies.

### BACKGROUND OF THE INVENTION

1. Definitions

For clearness, various terms relating to the biological molecules used hereinafter are defined in advance.

“Polymorphism” refers generally to the ability of an organism or gene to occur in two or more various forms. Particularly, for purposes of the present invention, “polymorphism” refers to two or more various forms of a same gene.

“Single Nucleotide Polymorphism” or “SNP” refers to a polymorphism that is due to a difference in a single nucleotide.

“Multiplex Polymerase Chain Reaction” or “MPCR” refers to the simultaneous amplification of multiple DNA target fragments in a single PCR reaction.

“High-throughput” refers to speedy and cost-effective production in large-scale manner. In the present invention, simultaneously screening a large number of various genetic loci within a single DNA sample pool can be routinely accomplished in a demanding time and budget.

2. Related Arts

Polymerase chain reaction (PCR) Polymerase chain reaction is one of the greatest achievements in science. Its widespread and versatile applications on producing large numbers of copies of DNA molecules from minute quantities of source DNA material have revolutionized the world of molecular biology. This method involves using paired sets of sequence-predetermined oligonucleotides or primers that anneal to their complementary DNA sequence and define the specified DNA fragment to be amplified by the aid of a thermostable DNA polymerase. The DNA products are synthesized through a repetitive series of cycles, each of which consists of template denaturation, primer annealing and extension of the annealed primers by a DNA polymerase, to create exponential accumulation of a specific fragment whose end are determined by the 5′ ends of the primers, see Saiki et al., “Primer-directed enzymatic amplification of DNA with a thermostable DNA polymerase”, Science (1988) 239:487-91.

Single Nucleotide Polymorphism (SNP) Genotyping

Several PCR variants have been developed for specific applications in diverse fields. The utilization of PCR on genotyping technology opens a broad window toward unraveling the mysterious veil of genome structure such that gene identification and discrimination becomes feasible in practice. Current enormous interest in SNPs demands genotyping technology advance to a much-improved level in which cost, accuracy, throughput and simplicity of assay design are key factors to determine such tasks executable. If 0.5 million SNPs would be analyzed in 1,000 individuals in a year, ca. 1.5 million SNP genotypes per day would be performed and the total cost would reach US $50 million. Moreover, the demanding of several mg genomic DNA, i.e., 10 or more blood collections per individual, also increases the cost on sample collection and makes it very impractical. What a haunting costs, though from a moderate estimate, would prohibit executing the project in even the largest genotyping center. Therefore, the demanding on high-throughput and cost-effective genotyping methods renders many innovative technologies, including PCR-derived techniques, to be developed; methods based on hybridization with allele-specific probes (e.g., TaqMan PCR method), oligonucleotides ligation (e.g., Oligonucleotide Ligation Assay), single nucleotide primer extension (e.g., MALDI-TOF Mass Spectrometry, FP-TDI), and enzymatic cleavage (e.g., Invader method) have been devised to either augment several automatic platforms or multiplex the biochemical genotyping reactions, see Syvanen, “Accessing genetic variation: genotyping single nucleotide polymorphisms”, Nat Rev Genet (2001) 2:930-42. However, none of them really represents a breakthrough on genotyping technology. Expensive instrumentation or regents, along with precious, but limited amount of genomic DNA samples available for large-scale SNP genotyping, are used to hinder their acceptance on the market and make the design of assay complicated. Moreover, huge consumption of sample DNA in most developed technologies prohibits large-scale SNP genotyping in practice.

Multiplex PCR

PCR multiplexing, the simultaneous amplification of two or more loci in a single PCR reaction, see Chamberlain, “Deletion screening of the Duchenne muscular dystrophy locus via multiplex DNA amplification”, Nucleic Acids Res (1988) 16:11141-56, is a powerful technique that considerably reduces the time and cost, as well as required genomic DNA samples, for genetic analysis. This method has been successfully applied to many areas of DNA testing, including determination of genetic polymorphisms; however, the pooling of a plurality of PCR primers in a single reaction could cause many problems, including increased formation of spurious PCR products and primer dimmers, and biased amplification of shorter DNA fragments. All these potential problems, if applied to SNP genotyping, lead to incorrect results. A detailed description about various conditions and encountered difficulties of multiplex PCR has been discussed by Henegariu et al., “Multiplex PCR: critical parameters and step-by-step protocol”, BioTechniques (1997) 23:504-511.

Problems of Prior Arts

As discussed in the above-related arts, there are at least three disadvantages not to be overcome, in summary, listed in the following.

Most currently available techniques for SNP genotyping require expensive instrumentation or regents, along with precious, but limited amount of genomic DNA samples available for large-scale SNP genotyping, and thus hinder their acceptance on the market and complicate the design of assay.

Multiplex PCR pooling multiple PCR primer pairs in a same reaction could cause many problems, including increased formation of spurious PCR products and primer dimmers, and enhanced amplification of shorter DNA fragments, and thus compromises its workable rate.

Due to the potential problems of multiplex PCR described above, the successful rate hardly reaches more than 50% from documentation as the technique was employed in SNP genotyping.

Several prior arts have been proposed to solve various problems in this field. For example, in U.S. Pat. No. 5,736,365, Walker et al. use multiplex Strand Displacement Amplification (SDA) in a single amplification reaction which is capable of simultaneously identifying M. tuberculosis and providing a screen for substantially all of the clinically relevant species of Mycobacteria. U.S. Pat. Nos. 5,882,856 and 6,207,372 issued to Shuber provide universal primer sequence for multiplex DNA amplification to allow multiplex PCR reactions to be designed and carried out without elaborate optimization steps, irrespective of the potentially divergent properties of the different primers used and to simultaneously produce equivalent amounts of each one of many amplification products. Diamandis et al. propose method, reagents and kit for diagnosis and targeted screening for p53 mutations, in U.S. Pat. No. 6,071,726, for rapid and cost effective diagnosis of p53 mutations in a sample of patients. These proposed methods did not deal with the above problems.

In U.S. Pat. Publication No. 20020058281, Matsuzaki et al. teach methods and compositions for multiplex amplification of nucleic acids, which permit the amplification of different sequences with the same efficiency so that approximately equimolar products result. This method needs high concentration of primers and uses single annealing temperature, and its PCR products are non-specific and its workable rate is very low. To solve the difficulty of using low amount of genomic DNA as template and higher number of pooled primer pairs in multiplex PCR, by use of hot start Taq polymerase in multiplexing amplification reactions, Nakamura et al. disclose a method for SNP typing in U.S. Pat. Publication No. 20020182622, which can genotype hundreds of thousands of SNP sites using a remarkably small amount of genomic DNA. However, this method still uses high concentration of primers and single annealing temperature, and by which the PCR products are non-specific and the workable rate is still low (not over 50%).

Accordingly, it is desired a high-throughput and cost-effective method for simultaneous amplification of target DNA sequences with high fidelity and workable rate.

### SUMMARY OF THE INVENTION

An object of the present invention is to provide a speedy and affordable method for simultaneous amplification of multiple target DNA sequences by multiplex PCR accompanying with conditional touchdown strategies. The invented method can be applied, but not limited, to SNP genotyping. Furthermore, according to the present invention, the required amount of DNA template applied to SNP genotyping is dramatically reduced compared with conventional genotyping methods.

The incorporation of touchdown PCR to multiplex PCR in the invented method is intended to solve the difficulty of misprimed PCR products encountered in PCR multiplexing. The touchdown PCR is a PCR variant that has been adopted to circumvent more complicated optimization processes for determining annealing temperature. It involves decreasing the annealing temperature by 1° C. every second cycle to a ‘touchdown’ annealing temperature, which is then used for 10 or so cycles. The spirit is that any differences in temperature Tm between correct and incorrect annealing gives a 2-fold difference in product amount per cycle, thus enriching for the correct product over any incorrect products, see Don et al., “Touchdown PCR to circumvent spurious priming during gene amplification”, Nucleic Acids Res (1991) 19:4008. The invented method that incorporates touchdown strategies to multiplex PCR is highly valuable when large number of primer pairs is required, especially in the case of SNP genotyping.

In particular, the invented method is devised to improve the performance on SNP genotyping in which stringent demanding on accuracy, cost-effective and high-throughput is a must. In addition, all the conventional processes for SNP genotyping require at least tens of nanograms (ng) of genomic DNA to genotype one SNP site. As hundreds or thousands of SNP sites per individual are required, it is unlikely to obtain enough amount of genomic DNA in practice. In contrast, simultaneous typing of multiple SNP sites by utilizing the invented method can greatly reduce the amount of genomic DNA required per individual, and thus, its potential cost on clinical sample collection. Moreover, its simplicity design of assay also makes the invented method highly desirable on laborious and tedious process of SNP genotyping.

In a conditional touchdown multiplex PCR, according to the present invention, a simultaneous PCR and a specific PCR are comprised. In the simultaneous PCR, the amplification is performed to increase the primers annealing to templates, and the specific PCR is to enrich the abundance of the designated sequence. Either one or both of the annealing temperatures for the simultaneous PCR and specific PCR employ a touchdown strategy. Particularly, the annealing temperature for the specific PCR is higher than that for the simultaneous PCR.

In a preferred embodiment of the present invention, a genotyping method comprises a simultaneous amplification step for the multiplex PCR with loose touchdown strategy (LTS), and a specific amplification step to the PCR products with stringent touchdown strategy (STS). An optimization step is further comprised before the amplifications for pre-adjusted primer pairs used in the multiplex PCR to improve the efficiency of the amplifications thereafter.

In the optimization step, for example a systematic primer optimization provides a method to increase workable rate in the following PCR reactions. The method utilizes touchdown PCR protocol and narrows the best descending gradient sections to 70-60° C. and 75-65° C. The primer pairs can be further pooled to these two gradient sections. In one implementation, 96 pairs of primers are pooled and the workable rate of multiplex PCR reaches 92.7%. In the following application on genotyping, the successful rate can reach 84.4%.

In PCR reactions, preferably, a thermostable Taq DNA polymerase plus a proof reading pfu DNA polymerase are used to increase the sequence fidelity of PCR products.

Simultaneous DNA amplification is conducted by utilizing multiplex PCR and a loose touchdown strategy such that an ensemble of target DNA sequences can be enriched through this step. In the simultaneous amplification of one implementation, 3° C. decrement of gradient temperature section for primer annealing, i.e., 67-57° C. and 72-62° C., is demonstrated to be an appropriate temperature decrement for exemplification. The enriched target sequences ensembles are ready for the following specific amplification after clean-up procedure.

The specific amplification of particular primers can amplify a certain target sequence by utilizing PCR and a stringent touchdown strategy. In one implementation, for the specific amplification, 3° C. increment of gradient temperature section for primer annealing, i.e., 73-63° C. and 78-68° C., is demonstrated to be an appropriate temperature increment for exemplification. The specific PCR products require further clean-up procedure and are subjected to other applications. In preferred embodiments, sequencing of PCR products is adopted to determine SNP sites. The successful rate for SNP discrimination in one implementation reaches 88.5% as 96 primer pairs pooled.

In another embodiment for fluorescence polarization/template-directed dye-terminator incorporation (FP-TDI) method, according to the present invention, the PCR primers are designed to have a melting temperature between 52° C. and 56° C. for amplification of 100 to 250 bp of PCR products, and the touchdown program is employed with 50-60° C. in the step of simultaneous PCR and 56-66° C. in the step of specific PCR.

Accordingly, it features a high-throughput, cost-effective and accurate method for demanding applications such as SNP genotyping and detection. The reduction of cost on clinical samples required for PCR multiplexing also benefits to large-scale genotyping. The ability of multiplexing 96 or more amplification in a PCR reaction with high sequence fidelity makes the application of the inventive method valuable, especially used in high-throughput SNP discoveries.

From one scope of the present invention, the problems solved includes at least:

the number limitation of pooling primer pairs for multiplex PCR;

the low workable rate of multiplex PCR due to the increased formation of spurious PCR products and enhanced amplification of shorter DNA fragments; and

the low successful rate of SNP genotyping as multiplex PCR techniques is employed.

Due to the various features, it is found advantageous to applications of the invented method for PCR-based genotyping.

### BRIEF DESCRIPTION OF THE DRAWINGS

These and other objects, features and advantages of the present invention will become apparent to those skilled in the art upon consideration of the following description of the preferred embodiments of the present invention taken in conjunction with the accompanying drawings, in which:

FIG. 1 shows the steps and the preferred touchdown strategies employed thereof according to the present invention;

FIG. 2 shows the annealing temperatures for three types of the touchdown strategies according to the present invention;

FIG. 3 shows a diagram illustrating the genotyping workflow according to the present invention, in which an optimization step, a simultaneous amplification step, and a specific amplification step are included;

FIG. 4 illustrates the gel analysis for the amplification results of an SNP genotyping;

FIG. 5 illustrates an exemplary sequence analysis upon specified region of fas-associated death domain (FADD) gene between conventional PCR only (FIG. 5A) and the invented genotyping method by using multiplex PCR with touchdown strategies (FIG. 5B);

FIG. 6 illustrates the gel analysis for the specific PCR in a multiplex-FP; and

FIG. 7 illustrates exemplary scatter plots for a multiplex-FP (FIG. 7A) according to the present invention and for a conventional simplex-FP (FIG. 7B), respectively.

### DETAILED DESCRIPTION OF THE INVENTION

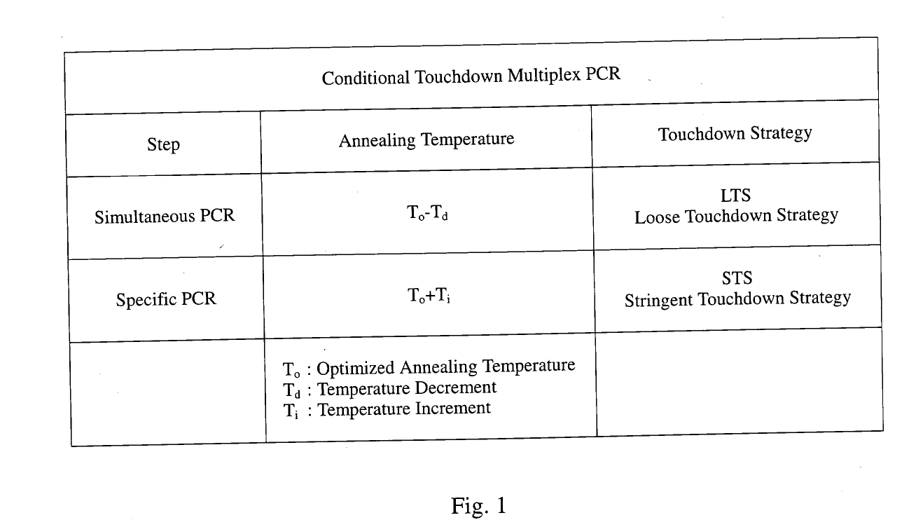

In a conditional touchdown multiplex PCR, according to the present invention, a simultaneous amplification step is performed to increase primers annealing to templates, followed by a specific amplification step to enrich the abundance of sequence of interest. Preferably, as shown in FIG. 1, both the simultaneous PCR and specific PCR employ touchdown strategies. Specifically, loose touchdown strategy (LTS) is incorporated in the simultaneous PCR for the purpose of dramatic amplification of primers annealing to DNA templates, which is performed with an annealing temperature lower than the optimized annealing temperature Tₒ by a temperature decrement T_{d}. In contrast, stringent touchdown strategy (STS) is for the specific PCR to specifically amplify the designated DNA sequences with an annealing temperature higher than the optimized annealing temperature Tₒ by a temperature increment T₁. This manner the combination of the simultaneous PCR and specific PCR with touchdown strategies achieves a speedy and affordable simultaneous amplification of multiple target DNA sequences.

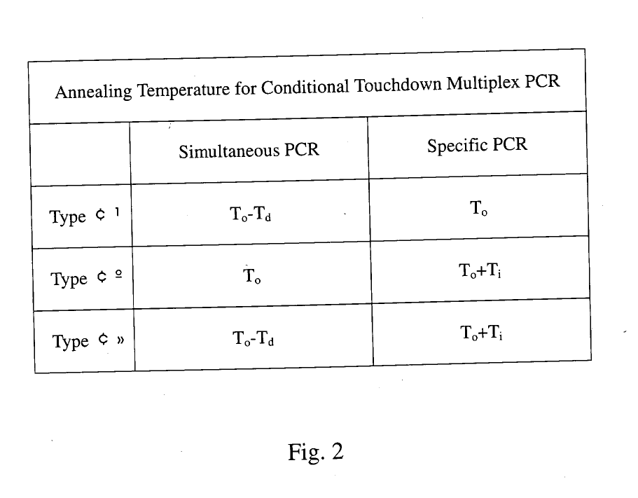

However, either one or both of the amplification steps performed with a touchdown strategy are available. In detail, there are two variations of the conditional touchdown multiplex PCR alternative to the embodiment shown in FIG. 1. As shown in FIG. 2, type ¢° is a loose touchdown simultaneous PCR and type ¢° is a stringent touchdown specific PCR, other than the embodiment of FIG. 1 designated with type ¢>>hereto.

Multi-Genotyping

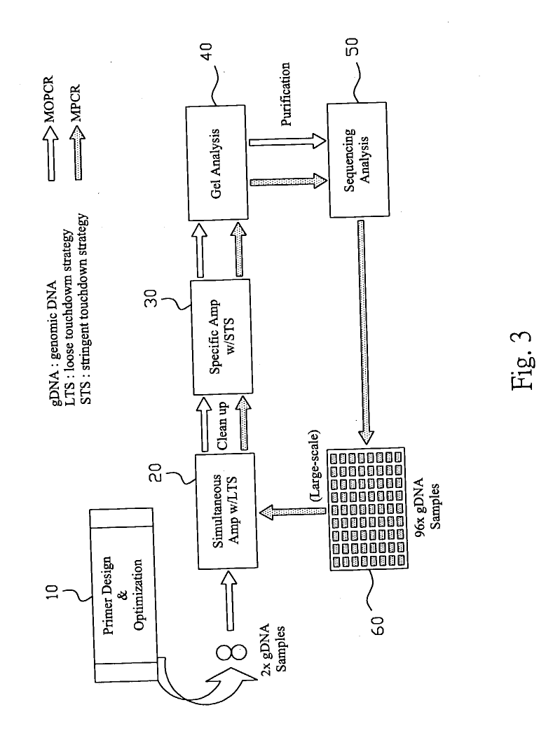

In the application of a typical genotyping according to the present invention, it is used multiple pairs of primers to co-amplify the multiple target sequences, for example multiple SNP sites using genomic DNA to be analyzed, which is accomplished by three steps: (1) an optimization step for pre-adjusted primer pairs, (2) a simultaneous amplification step for a multiplex PCR to the primer pairs with loose touchdown strategy, and (3) a specific amplification step to the multiple PCR products with stringent touchdown strategy. An explanatory workflow is depicted in FIG. 3. This diagram illustrates a preferred genotyping workflow incorporating the inventive conditional touchdown multiplex PCR that comprises an optimization step 10, a simultaneous amplification step 20, and a specific amplification step 30. In the standard optimization procedure (MOPCR), two genomic DNA samples and pooled multiple primer pairs are subjected to amplification steps with various touchdown-thermal cycling profiles so as to optimize best multiplex PCR conditions. Furthermore, gel analysis 40 and sequencing analysis 50 are adopted to validate PCR and genotyping results. After optimization of PCR conditions, large-scale multiplex PCR is processed on 96-well plates 60, each well including a genomic DNA sample from specific individual. The results are resolved by gel and sequencing analyses 40 and 50. The core technology of this method comprises the simultaneous and specific amplification steps 20 and 30 with loose touchdown strategy (LTS) and stringent touchdown strategy (STS), respectively. Compared with conventional multiplex PCRs, this method using loose and stringent touchdown strategies increases workable rate for multiple PCR, successful rate in later application, DNA sequencing, and sequence fidelity of PCR amplicons.

1. Optimization Step

Preferably, the primers used in multiplex PCR are well designed based on multiple pre-adjusted parameters, including product size, oligonucleotide size, Tₘ value, GC contents and other parameters, for example shown in Table 1. Sophisticated software for primer designs is recommended to process designing tens or hundreds of primers. In preferred embodiments, Primer3 (MIT, MA) is utilized.

TABLE 1

| Parameter | Adjusted Details |
|---|---|
| Included Region | 200 bp_exon_200 bp |
| Product Size | 750-950 bp |
| Oligo Size | 21, 23, 25 (Min, Optima, Max) |
| Tₘ | 55, 60, 65 (Min, Optima, Max) |
| Max Tₘ Difference | 5 |
| GC % | 45, 50, 60 (Min, Optima, Max) |
| Max Self Complementarity | 6 |
| Max 3′ Complementarity | 3 |
| Max Poly-X | 5 |
| Mispriming Library | Human |
| 3′ end of Primer | No T residues |

The optimization step is accomplished via performing PCR by using one genomic DNA template and each primer pair. The cycling profile for touchdown PCR, so called “touchdown program”, includes: (1) template denaturing at 94° C. for 4 minutes; (2) ten cycles of 10° C. descending gradient of primer annealing temperature at 1° C./cycle, each of which includes 40 seconds of template denaturing at 94° C., followed by 40 seconds of primer annealing at designated point of gradient temperature, and then 3 minutes of primer extension at 72° C.; (3) 25 cycles of 40 seconds of template denaturing at 94° C., followed by 40 seconds of primer annealing at touchdown point of gradient temperature, and then 3 minutes of primer extension at 72° C.; (4) primer extension at 72° C. for 5 minutes. To optimize the best conditions for multiplex PCR reactions using touchdown program, several descending gradient sections for annealing temperature are tested to determine the best gradient section for multiplex PCR. In preferred embodiments, these gradient sections include 60-50° C., 65-55° C., 70-60° C. and 75-65° C. In the exemplary process, 70-60° C. covers the most sections best for diverse multiplex PCR reactions. Preferably, pairs of primers demonstrated to be the best performance in certain gradient section by this optimization step are pooled together and used in later multiplex PCR.

2. Simultaneous Amplification Step

In the step of simultaneous amplification 20, the interested DNA fragments are simultaneously amplified by introduction of multiple pairs of primers flanking the target sequences to each single PCR reaction. In this example, 96 primer pairs were pooled and used in processing multiplex PCR in 96-well plates, each well containing one individual genomic DNA sample. In order to increase the possibility that target sequences could simultaneously be annealed by their specific primer pairs and co-amplified, a loose condition, i.e., loose touchdown strategy or LTS, in which a few lower degrees of gradient temperature section for primer annealing is adopted. In preferred embodiments, 3° C. decrement of gradient temperature section, i.e., 67-57° C. and 72-62° C., is demonstrated to be an appropriate temperature decrement.

The PCR reactions are conducted by using loose touchdown strategy and the employed reagents are adjusted to appropriate conditions. In preferred embodiments, a thermostable DNA polymerase (e.g., Taq DNA polymerase) plus pfu DNA polymerase (a proof reading DNA polymerase) are used and the concentrations of KCl and MgCl₂ in the buffer are adjusted to 1.5 fold compared with manufacture's suggestion. Alternatively, TPC Taq DNA polymerase (final conc.: 0.04 U/μl; Taiwan Proteomics Company, Taiwan), pfu DNA polymerase (final conc.: 0.0005 U/μl; Stratagene, Calif.), 1.5 fold of manufacture's 10×PCR buffer (100 mM Tris-HCl PH9.0, 15 mM MgCl₂, 500 mM KCl, 1% gelatin and 1% Triton X-100 included), 0.25 mM dNTP, 0.025 or 0.05 μM of each primer and 25-50 ng genomic DNA are employed in other embodiments of the present invention. The thermal cycling profile for simultaneous amplification by the multiplex PCR has been described as in the part of the optimization step 20 with prolonged extension time and loose touchdown program. All the PCR products are subjected to clean up by either 70% ethanol or other clean-up filtration systems. Preferably, Multi-screen PCR 96-well filtration system (Millipore, Mass.) is utilized. After clean-up, the PCR products are ready for next step of specific amplification. The PCR products in each reaction possess a collection of target DNA sequences, for example an ensemble of interested SNP sites, from an individual.

3. Specific Amplification Step

In the step of specific amplification 30, specific primer pairs are utilized and advanced to amplify particular target sequence in each PCR reaction. A stringent condition, stringent touchdown strategy or STS, in which a few higher degrees of gradient temperature section for primer annealing is adopted such that the specificity of primer annealing to template could be achieved. In preferred embodiments, likewise, 3° C. increment of gradient temperature section, i.e., 73-63° C. and 78-68° C., is demonstrated to be an appropriate temperature increment.

The details of the PCR reaction herewith is the same as described in the optimization step 10 except that DNA template is from the product of the amplification step 20 and a stringent touchdown program is used thereof. In addition, the time for primer extension in each amplification cycle reduces to 1 minute and 30 seconds, instead 3 minutes in the simultaneous amplification step. In preferred embodiments, 1/150 quantity of simultaneous amplification PCR products is subjected to PCR reaction. The clean-up step is required for final PCR products if further application is necessary. For example, the final PCR products are subjected to DNA sequencing by Applied Biosystems DNA analyzer 3700 from Applied Biosystems at California for the purpose of SNP genotyping.

[Experiment]

The following data are provided to describe the above DNA genotyping in more illustrated manner, but the technical scope of the present invention is not limited to the examples.

In an embodiment of the present invention, 96 genomic DNA samples are collected from the University Hospital of National Taiwan University. A detailed description for the usage of the collected samples is well informed and the consent documents are acquired from each individual. All the genomic DNA samples are purified by standard procedure and stored at −20° C. before use. To reach the best conditions for multiplex PCR, two genomic DNA samples are first used in the optimization procedure 10. In the simultaneous amplification step 20, 96 primer pairs are pooled in 100 μl of PCR reagents comprising the same composition described in the foregoing description. The detailed procedure for manipulating PCR reactions with loose touchdown strategy is also described therewith. In the step of specific amplification 30, 96 primer pairs, each of which is designed for typing specific genetic loci, are individually subjected to each well of 96-well plate. 1/150 of PCR products from the simultaneous amplification step 20 are served as the templates in each parallel PCR reaction with stringent touchdown strategy. The resulting PCR products are resolved by 2% agarose gel and analyzed by Applied Biosystems DNA analyzer 3700 to validate genotypes. In both simultaneous and specific amplifications 20 and 30, PCR products are subjected to Millipore Multi-screen PCR 96-well filtration system for clean-up.

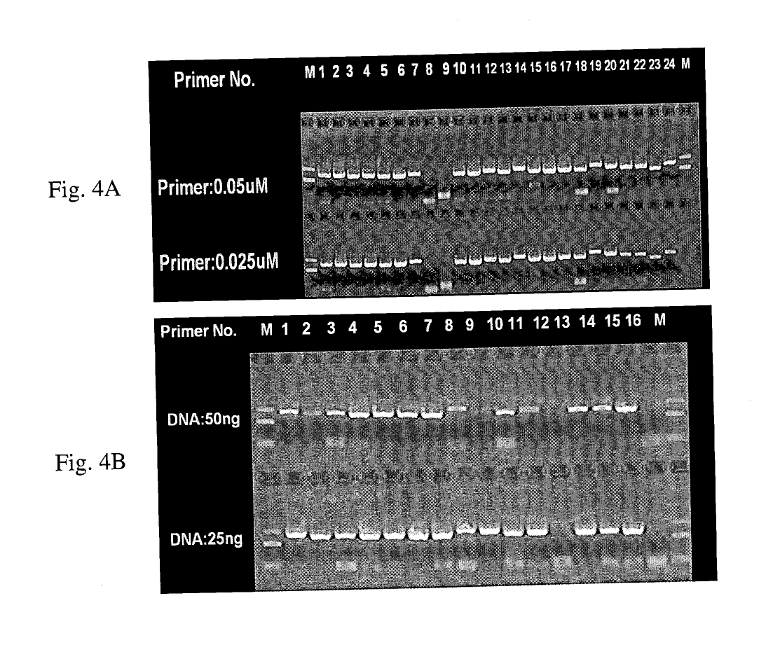

FIG. 4 illustrates part of the results from the specific amplification step 30. 24 of 96 PCR products are illustrated in FIG. 4A, where two concentrations of primers, 0.05 μM and 0.025 μM, are applied in the specific amplification step 30. No difference between using these two concentrations of primers is observed in the gel, even using primer concentration as low as 0.025 μM. On the other hand, in FIG. 4B, 50 and 25 ng of genomic DNA served as template in the simultaneous amplification step 20 are demonstrated that lower amount of genomic DNA, 25 ng hereof, can reach higher workable rate in multiplex PCR. In other words, 25 ng of genomic DNA served as template in the simultaneous amplification step 20 is found to increase workable rate of specific amplification, compared with using 50 ng genomic DNA in the same method. The date thereof demonstrates that tiny amount of genomic DNA, 25 ng or lower, is required for genotyping according to the present invention.

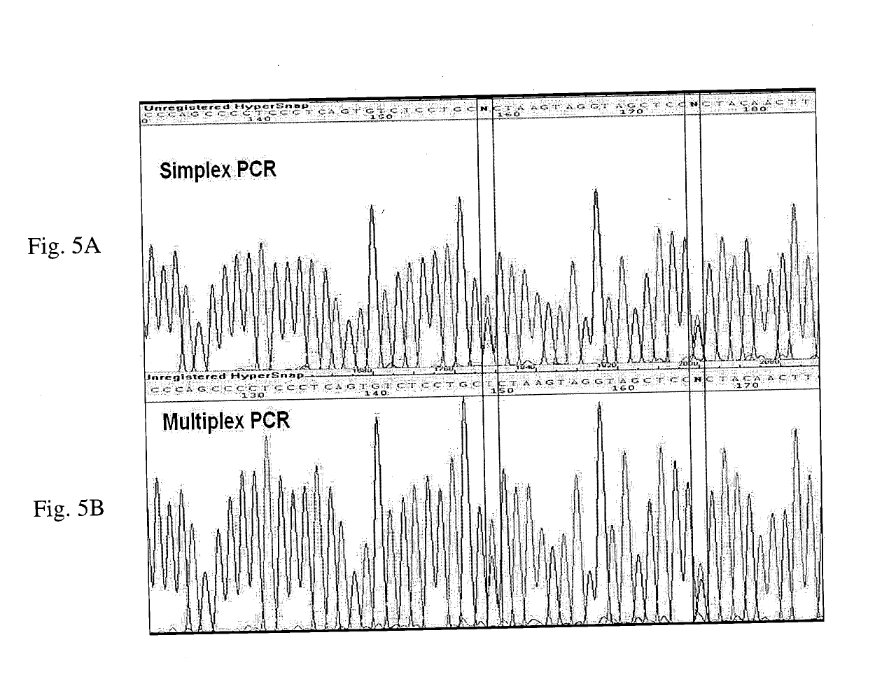

FIGS. 5A and 5B are provided to show one example of consistency between the methods of SNP genotyping by using conventional PCR only and multiplex PCR with LTS and STS, respectively. The consistency of sequencing pattern is shown in FIGS. 5A and 5B, respectively, with enclosed rectangles to indicate SNP loci thereof. No preference of typing certain genotypes, as well as no difference between the patterns in both sequencing results, are found as the invented multi-genotyping method is used. Except for this, 50 cases of SNP loci have been examined and found no preference of certain genotypes were typed by using either method.

FP-TDI

Although the FP-TDI assay is one of several different methods that are currently available for automated genotyping, it has a number of advantages, such as ease-of-use, robust performance and affordable cost. It is also an excellent application of the invented method. Below is a practiced experiment to illustrate the features of the present invention.

1. Primer Design

The PCR primers are designed to have a melting temperature between 52° C. and 56° C. for amplification of 100 to 250 bp of PCR products.

2. Multiplex PCR Amplification

In the same as previous description but with 60 to 50° C. touchdown program in the step of simultaneous PCR and 66 to 56° C. touchdown program in the step of specific PCR.

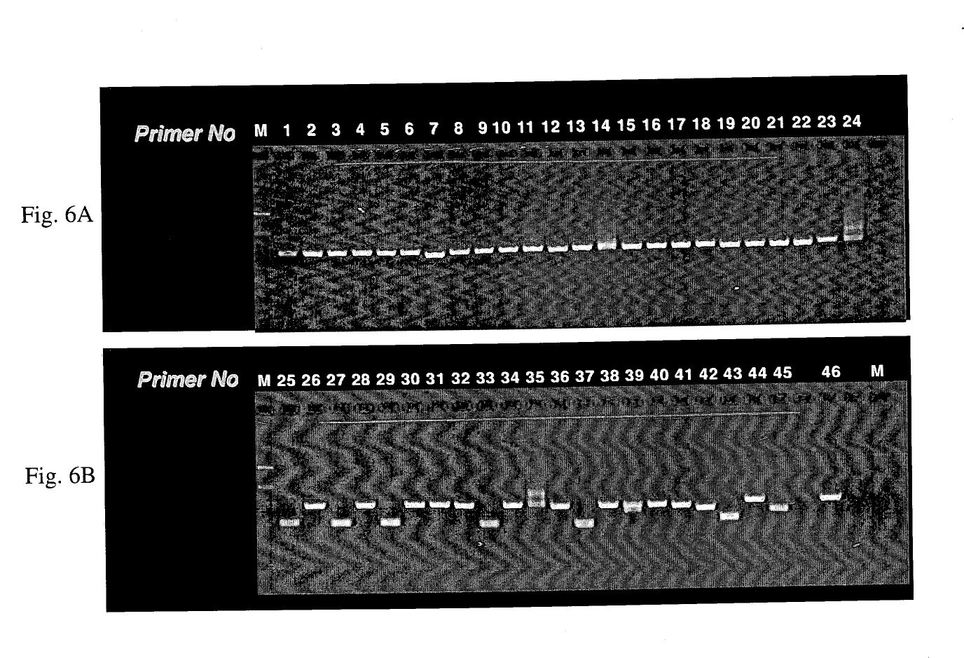

FIG. 6 is the gel analysis for the specific PCR in a multiplex-FP with 46 primer pairs, and its workable rate is nearly 89.1%.

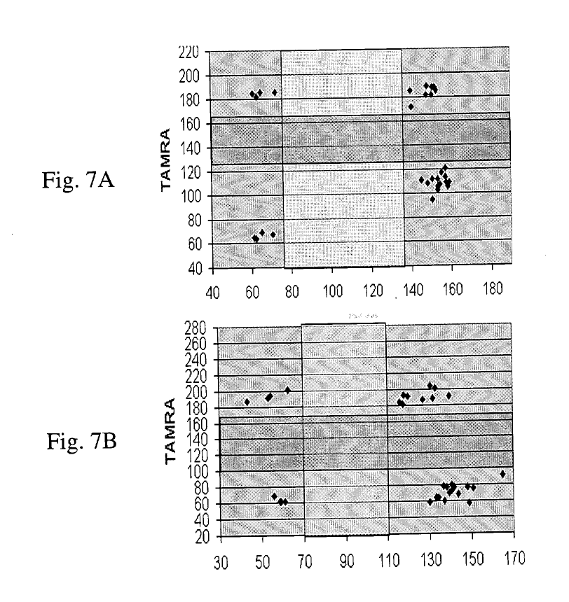

FIG. 7A shows a scatter plot for a multiplex-FP according to the present invention and FIG. 7B shows a scatter plot for a conventional simplex-FP.

The incorporation of conditional touchdown strategies to multiplex PCR herewith is first proposed and demonstrated to be beneficial in solving the difficulty of misprimed PCR products encountered in the technique of PCR multiplexing as employed in genotyping. The efficacious number of pooling primers in multiplex PCR, unlike no more than 10 pairs in conventional methods, could reach to at least 96 primer pairs. When the invented method applied to disease diagnosis, genes at least up to 96 sites in the described example, unlike one or several genes in conventional methods, could be discriminable. In addition, low quantity of genomic DNA samples is required in assays, compared with other methods for large-scale genotyping. Moreover, the features of high-throughput and cost-effective do not jeopardize the simplicity of the assay design, compared with other genotyping methods equipped with expensive and complicated instrumentation and reagents.

Potential Applications

The invented method can be applied, but not limited, to different aspects as following:

large-scale and high-throughput genotyping from small amount of gDNA;

the screening kits for multi-disease/complex disease;

the diagnosis kits for genetic and infectious diseases; and

the prognostic kits for drug efficacy.

While the present invention has been described in conjunction with preferred embodiments thereof, it is evident that many alternatives, modifications and variations will be apparent to those skilled in the art. Accordingly, it is intended to embrace all such alternatives, modifications and variations that fall within the spirit and scope thereof as set forth in the appended claims.

What is claimed is:

1. A conditional touchdown multiplex PCR comprising the steps of:
a simultaneous PCR with a first temperature for increasing a plurality of primers annealing to templates; and
a specific PCR with a second temperature for enriching a plurality of designated sequences;
wherein said second temperature is higher than said first temperature.

2. A conditional touchdown multiplex PCR according to claim 1, wherein said first temperature equals substantially to an optimized annealing temperature and said second temperature is higher than said optimized annealing temperature by a temperature increment.

3. A conditional touchdown multiplex PCR according to claim 1, wherein said first temperature is lower than an optimized annealing temperature by a temperature decrement and said second temperature equals substantially to said optimized annealing temperature.

4. A conditional touchdown multiplex PCR according to claim 1, wherein said first temperature is lower than an optimized annealing temperature by a temperature decrement and said second temperature is higher than said optimized annealing temperature by a temperature increment.

5. A conditional touchdown multiplex PCR according to claim 1, further comprising optimizing primer pooling and associated PCR conditions before said simultaneous PCR.

6. A genotyping method comprising the steps of:
simultaneously amplifying multiple nucleotide sequences in a pool with a plurality of primer pairs and genomic DNA by a multiplex PCR with a first touchdown program; and
multiplexing individual PCR reactions with a second touchdown program, each of said individual PCR reactions amplifying a specific nucleotide sequence flanked by a pair of designated primers from multiplex PCR products produced in the previous step.

7. A method according to claim 6, wherein said first touchdown program uses a temperature substantially equal to an optimized annealing temperature and said second touchdown program uses another temperature higher than said optimized annealing temperature by a temperature increment.

8. A method according to claim 6, wherein said first touchdown program uses a temperature lower than an optimized annealing temperature by a temperature decrement and said second touchdown program uses another temperature substantially equal to said optimized annealing temperature.

9. A method according to claim 6, wherein said first touchdown program uses a temperature lower than an optimized annealing temperature by a temperature decrement and said second touchdown program uses another temperature higher than said optimized annealing temperature by a temperature increment.

10. A method according to claim 6, wherein said genomic DNA have an amount smaller than 50 ng.

11. A method according to claim 6, wherein said genomic DNA have an amount equal to or more than 50 ng.

12. A method according to claim 6, wherein said step of simultaneously amplifying multiple nucleotide sequences employs a touchdown-thermal cycling profile.

13. A method according to claim 12, wherein said cycling profile comprises:
a first template denaturing;
a plurality of first cycles of a descending gradient of a primer annealing temperature, each including a first time duration of a second template denaturing, followed by a second time duration of a first primer annealing at a designated point of gradient temperature, and then a third time duration of a first primer extension;
a plurality of second cycles of a fourth time duration of a third template denaturing, followed by a fifth time duration of a second primer annealing at a touchdown point of gradient temperature, and then a sixth time duration of a second primer extension; and
a third primer extension.

14. A method according to claim 6, further comprising a step of cleaning up said multiple PCR products before said step of multiplexing individual PCR reactions.

15. A method according to claim 6, wherein said step of multiplexing individual PCR reactions employs a touchdown-thermal cycling profile.

16. A method according to claim 15, wherein said cycling profile comprises:
a first template denaturing;
a plurality of first cycles of a descending gradient of a primer annealing temperature, each including a first time duration of a second template denaturing, followed by a second time duration of a first primer annealing at a designated point of gradient temperature, and then a third time duration of a first primer extension;
a plurality of second cycles of a fourth time duration of a third template denaturing, followed by a fifth time duration of a second primer annealing at a touchdown point of gradient temperature, and then a sixth time duration of a second primer extension; and
a third primer extension.

17. A method according to claim 6, further comprising a step of optimizing primer pooling and associated PCR conditions before said step of simultaneously amplifying multiple nucleotide sequences.

18. A method according to claim 6, further comprising a gel analysis and a sequencing analysis adopted to validate PCR and genotyping results.

19. An FP-TDI method comprising the steps of:
simultaneously amplifying multiple nucleotide sequences in a pool with a plurality of primer pairs and genomic DNA by a multiplex PCR with a first touchdown program; and
multiplexing individual PCR reactions with a second touchdown program, each of said individual PCR reactions amplifying a specific nucleotide sequence flanked by a pair of designated primers from multiplex PCR products produced in the previous step.

20. A method according to claim 19, wherein said first touchdown program uses a temperature substantially equal to an optimized annealing temperature and said second touchdown program uses another temperature higher than said optimized annealing temperature by a temperature increment.

21. A method according to claim 19, wherein said first touchdown program uses a temperature lower than an optimized annealing temperature by a temperature decrement and said second touchdown program uses another temperature substantially equal to said optimized annealing temperature.

22. A method according to claim 19, wherein said first touchdown program uses a temperature lower than an optimized annealing temperature by a temperature decrement and said second touchdown program uses another temperature higher than said optimized annealing temperature by a temperature increment.

23. A method according to claim 19, further comprising a step of designing said primers to have a melting temperature in a range for amplification of a quantity of PCR products.
